(19) United States
(12) Patent Application Publication
(10) Pub. No.: US 2024/0366365 A1
(43) Pub. Date: Nov. 7, 2024

(54) SURGICAL METHOD AND IMPROVED SUTURES FOR HEART VALVE REPLACEMENT

(71) Applicant: Badr Idbeis, Wichita, KS (US)

(72) Inventor: Badr Idbeis, Wichita, KS (US)

(21) Appl. No.: 18/309,885

(22) Filed: May 1, 2023

(51) Int. Cl. A61F 2/24; A61B 90/92

(52) U.S. Cl. CPC A61F 2/2409; A61B 90/92; A61B 2017/0406; A61B 2017/06057

(57) ABSTRACT

A surgical heart valve replacement method with improved sutures for reducing the propensity for surgeons to overtighten or undertighten the sutures when securing an artificial heart valve to a native valve annulus.

### BACKGROUND

Valvular heart disease such as aortic stenosis (AS) is becoming more common as populations age in many countries. Surgical valve replacement with either a mechanical or a biological valve is currently the gold standard of care for patients with severe valvular heart disease. Sewed-in, stented bioprosthetic valves are the most commonly implanted devices, especially in elderly patients. They typically consist of a plastic and/or metal frame that is coated with fabric or tissue and that supports leaflets made of xenograft soft tissue. At the base of the valve, a sewing ring covered in fine fabric is attached.

Pledget-armed sutures are commonly used to secure an artificial heart valve to a native valve annulus in either an intra-or supra-annular position. In one popular attachment procedure, sutures each having a thread with needles on both ends and a pledget intermediate the needles are used. Both needles of a suture are passed through a native valve annulus and then passed through the sewing ring of the artificial valve. After all of the sutures are placed in this manner, the loose ends of the sutures are tightened to firmly seat their pledgets against the native valve annulus and then tied.

Unfortunately, the above-described attachment procedure and similar procedures can lead to problems. As a suture is tightened to seat its corresponding pledget, the pledget is pulled behind the artificial valve where it can't be seen by the surgeon, so the seating process is partially blind. Surgeons also have difficulty in distinguishing the many loose ends of the sutures from one another because the sutures are closely spaced. Surgeons therefore often have difficulty in determining which suture ends belong to which pledget. These issues often lead to either overtightening or undertightening of sutures. Overtightening occurs when a surgeon continues to pull on the loose ends of a suture to seat its corresponding pledget when the pledget has actually already been seated. This happens because the surgeon may be looking at the wrong pledget when the correct pledget has already been seated and is therefore no longer visible. In this case, the surgeon may continue pulling on the already tightened suture, causing the seated pledget and thread to pull through or otherwise damage the native annulus. Undertightening occurs when a surgeon stops pulling on the loose ends of a suture when the surgeon believes it has been properly seated when its pledget is in fact still loose and not properly seated. This happens because the surgeon may believe a pledget is out of view and seated but is looking for the wrong pledget when the correct pledget is still loose and not securely seated. Both suture overtightening and undertightening can jeopardize a valve replacement procedure and lead to significant surgical and post-surgical problems.

### SUMMARY

The present invention solves the above-described problems and provides a distinct advance in the art of heart valve replacement surgery. More particularly, the present invention provides a surgical heart valve replacement method that uses improved sutures that reduce the propensity for surgeons to overtighten and/or undertighten the sutures when securing an artificial heart valve to a native valve annulus.

The surgical method of the present invention overcomes the above-described problems by using at least first and second distinct sets of sutures. Each suture from the first set of sutures includes a thread with two ends, a first needle secured to the first end of the thread, a second needle attached to the second end of the thread, and a pledget. Importantly, the thread and pledget from each suture in the first set of sutures have a first distinguishing feature. In other words, the thread and pledget of each suture are matched in some way.

Each suture from the second set of sutures also includes a thread with two ends, a first needle secured to the first end of the thread, a second needle attached to the second end of the thread, and a pledget. As with the first set of sutures, the thread and pledget from each suture in the second set of sutures have a second distinguishing feature.

In one embodiment, the first and second distinguishing features are colors, such that the primary difference between the first and second sets of sutures is that their threads and pledgets have different colors. For example, the threads and pledgets of the sutures in the first set of sutures may be white, and the threads and pledgets of the sutures in the second set of sutures may be blue.

A cardiac surgeon may use the above-described sutures to attach an artificial heart valve to a native valve annulus as follows. After a patient's heart is exposed and a defective native heart valve is removed, the artificial heart valve is positioned near the native valve annulus. To attach the artificial heart valve to the native valve annulus, a suture from the first set of sutures is attached to the native valve annulus by passing both needles of the suture through the native valve annulus. The needles are then passed through the sewing ring of the artificial valve. A suture from the second set of sutures is then attached to the native valve annulus adjacent to the prior suture in the same manner by passing both needles of the suture through the native valve annulus and then through the sewing ring of the artificial valve. These steps are repeated with alternating sutures from the first and second sets of sutures such that adjacently positioned sutures are from different sets of sutures. After all of the sutures are placed, the loose ends of the sutures are tightened to firmly seat their pledgets against the native valve annulus and then tied.

Advantageously, the alternating sutures from the first and second sets of the sutures allow the cardiac surgeon to more easily distinguish the sutures and determine which suture ends belong to which pledgets. For example, if the surgeon tightens the ends of a blue suture thread, the surgeon knows to look for and monitor the seating of a blue pledget and not the two adjacent white pledgets. Similarly, if the surgeon tightens the ends of a white suture thread, the surgeon knows to look for and monitor the seating of a white pledget and not the two adjacent blue pledgets. This not only prevents the above-described overtightening and undertightening problems but also allows surgeons to proceed more quickly by spending less time trying to match suture threads with their pledgets.

Other embodiments of the sutures may have distinguishing features other than color. For example, the first set of sutures may have threads and pledgets with a first distinguishing pattern such as stripes, and the second set of sutures may have threads and pledgets with a second distinguishing pattern such as dots. In yet another embodiment, the threads and pledgets of the first set of sutures may have a pattern such as stripes or dots, and the threads and pledgets of the second set of sutures may have a solid color with no pattern. Thus, the present invention is intended to encompass sets of sutures which are distinct from one another such that the first set of sutures can be easily distinguished from the second set of sutures.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a perspective view of a first set of sutures that may be used in the present invention.

FIG. 2 is a perspective view of a second set of sutures that may be used in the present invention.

FIG. 3 is an enlarged view of one of the sutures from the first set of sutures.

FIG. 4 is an enlarged view of one of the sutures from the second set of sutures.

FIG. 5 is a schematic diagram showing attachment of an artificial valve to a patient's heart with the sutures of the present invention.

FIG. 6 is a flow diagram depicting exemplary steps of a method of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION

The present invention provides a surgical method and improved sutures that are used in the surgical method. The surgical method and sutures may be used for aortic valve replacement, mitral valve replacement, or any other surgery in which a medical device is attached to a patient. The surgical method and sutures reduce the propensity for surgeons to overtighten and/or undertighten the sutures when securing an artificial heart valve to a native valve annulus or when performing other surgeries.

Embodiments of the present invention include at least two distinct sets of sutures, with each set of sutures including any number of sutures needed by a surgeon for a heart valve replacement surgery or other surgery.

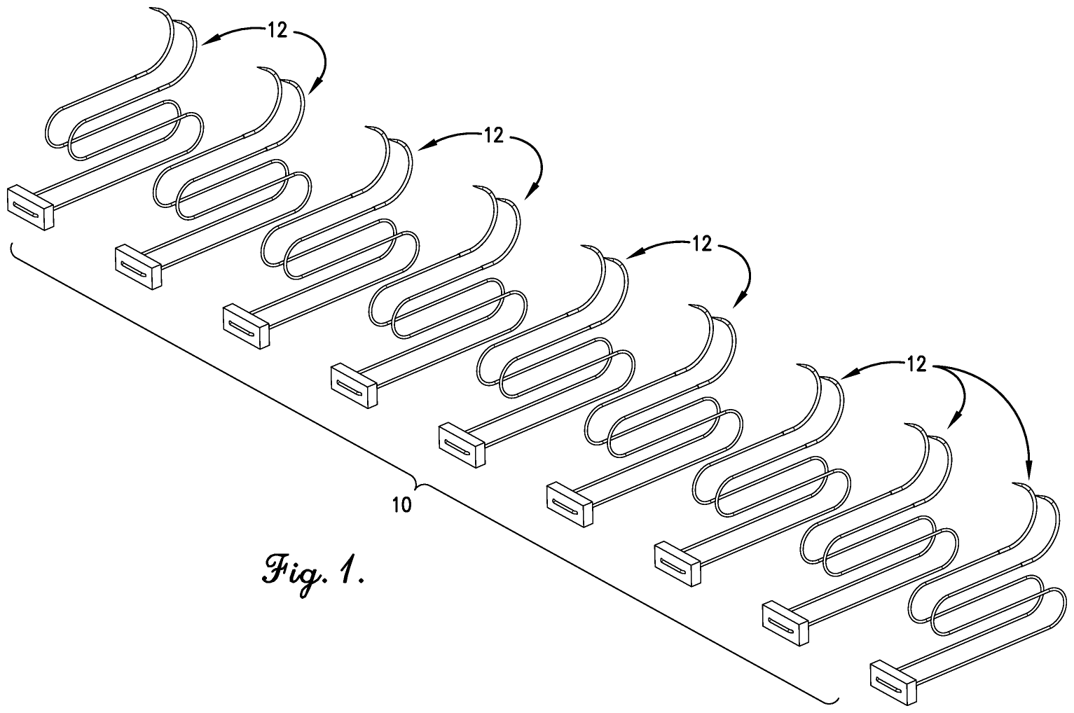

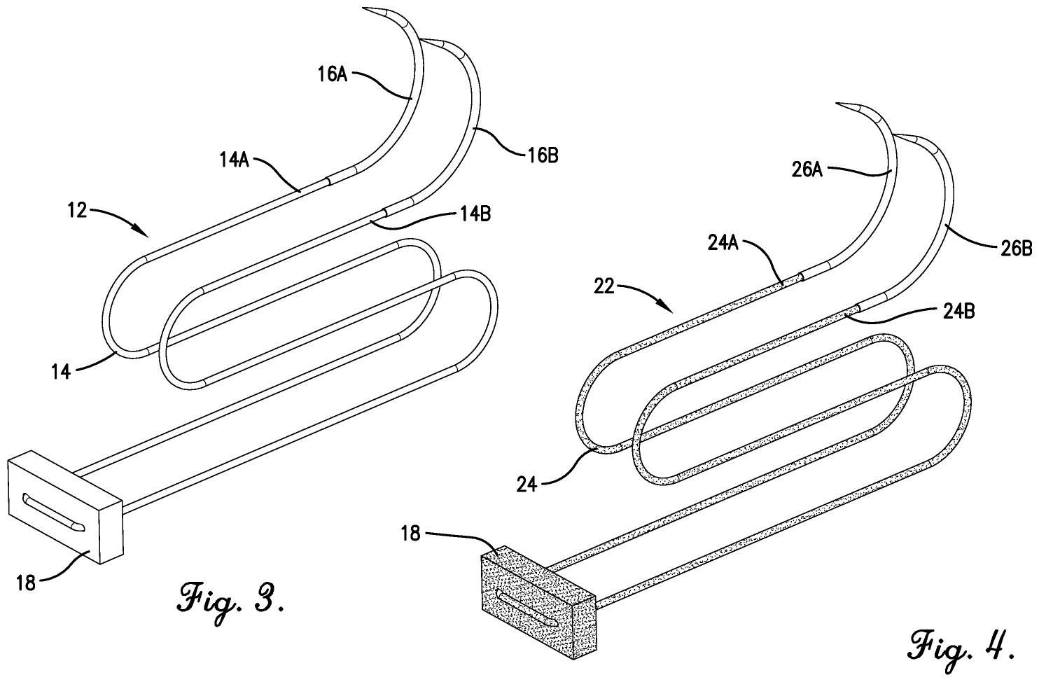

An exemplary first set of sutures 10 is shown in FIG. 1 and includes a number of identical sutures 12. As shown in FIG. 3, each suture 12 has a surgical thread 14 with two ends 14A, B, a first needle 16A secured to the first end of the thread, a second needle 16B attached to the second end of the thread, and a pledget.

The thread 14 of each suture 12 is a conventional surgical thread and may be made of synthetic materials and may be any length and diameter. The needles 16A, B of each suture 12 are conventional surgical needles and may be any shape and size commonly used for cardiac surgeries. In some embodiments, each suture may only include one needle. The pledget 18 of each suture 12 is a relatively thin and small pad which includes two openings through which the thread 14 is positioned. The pledget is roughly midway along the length of the thread. Importantly, the thread 14 and pledget 18 of each suture 12 in the first set of pledgets 10 are matched with a common first distinguishing feature. In one embodiment, the first distinguishing feature is a color. For example, the threads and pledgets of the first set of pledgets may be all white.

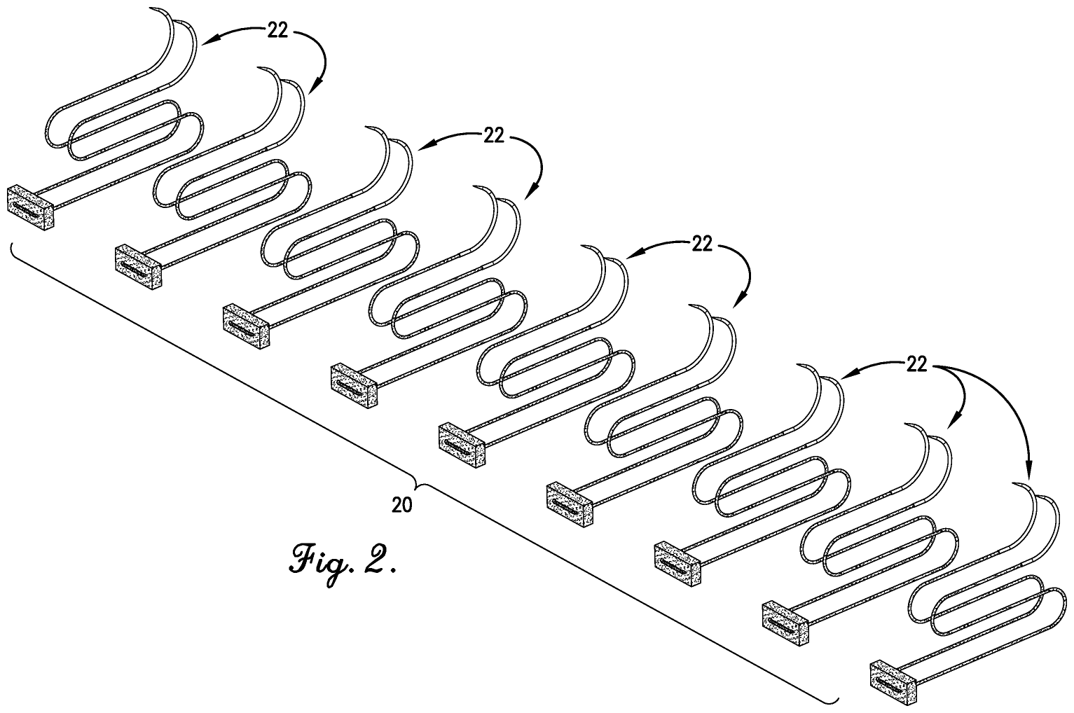

A second set of sutures 20 is shown in FIG. 2 and also a number of substantially identical sutures 22. As best illustrated in FIG. 4, each suture 22 has a surgical thread 24 with two ends 24A, B, a first needle 26A secured to the first end of the thread, a second needle 26B attached to the second end of the thread, and a pledget 28. In some embodiments, each suture may only include one needle.

Importantly, the thread 24 and pledget 28 of each suture 22 in the second set of pledgets 20 are matched with some distinguishing feature. In one embodiment, the threads and pledgets of the second set of pledgets are all blue. Thus, in this embodiment, the only significant difference between the first and second sets of sutures is color. Specifically, the thread and pledget of each suture in the first set of sutures has the same first color, such as white, and the thread and pledget of each suture in the second set of sutures has the same second color, such as blue.

Other embodiments of the of the first and second sets of sutures 10, 20 may have distinguishing features other than color. For example, the first set of sutures may have threads and pledgets with a common pattern such as stripes that serve as their first distinguishing feature, and the second set of sutures may have threads and pledgets with another pattern such as dots that serve as their second distinguishing feature. In yet another embodiment, the threads and pledgets of the first set of sutures pledgets may have a pattern such as stripes or dots, and the threads and pledgets of the second set of sutures may have a solid color with no pattern. Thus, the present invention is intended to encompass any two sets of sutures which are distinct from one another such that sutures from the first set of sutures can be easily distinguished from sutures from the second set of sutures. In yet other embodiments, more than two distinct sets of sutures may be used. For example, three sets of distinct sutures may be used, wherein the threads and pledgets in the first set of sutures may be white; the threads and pledgets in the second set of sutures may be blue; and the threads and pledgets in the third set of sutures may be orange. This further spaces like-colored threads and pledgets so a surgeon can even better distinguish the sutures.

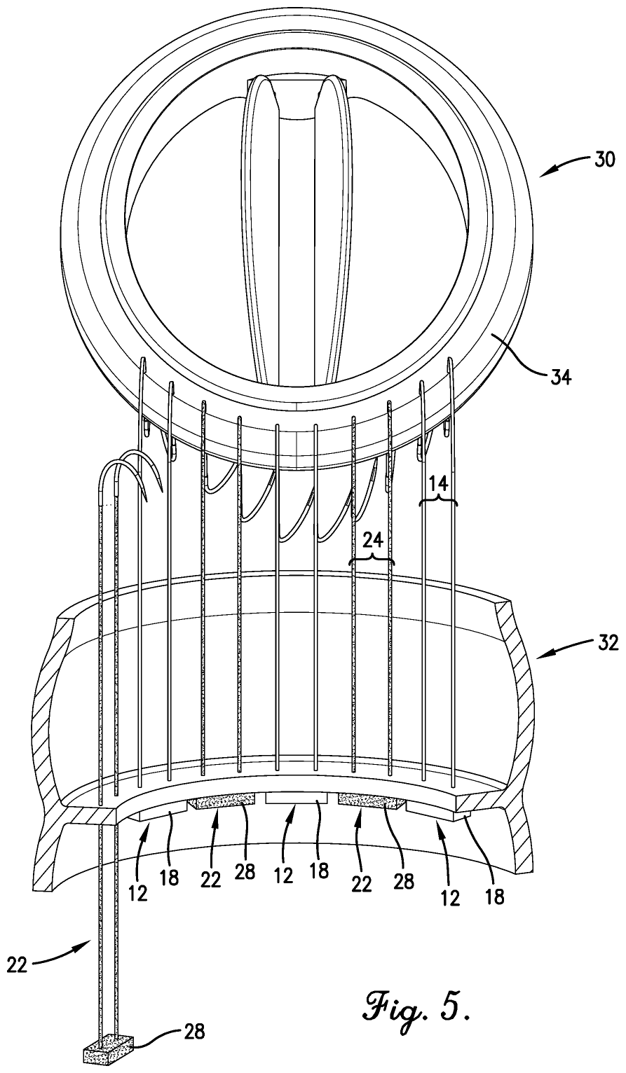

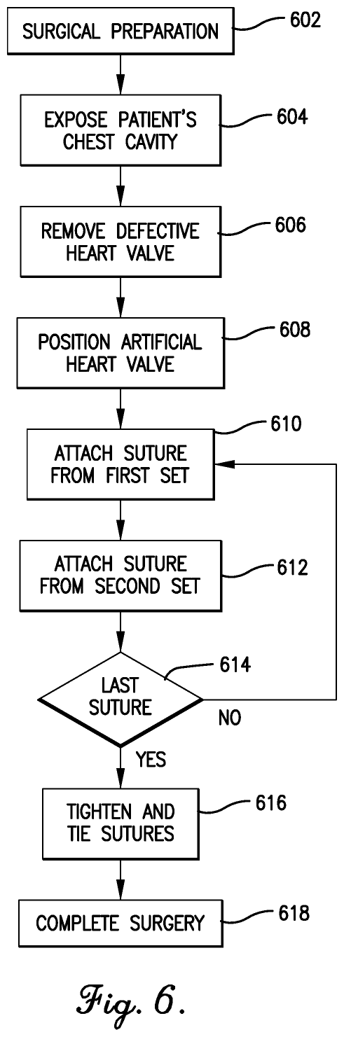

As depicted in FIG. 5, a cardiac surgeon may use the sutures 12, 22 described above and equivalent sutures to attach an artificial heart valve 30 to a native valve annulus 32. Although the sutures are particularly helpful for cardiac surgeries, they may also be used in other types of surgeries where it is difficult to distinguish adjacent sutures.

The flow chart of FIG. 6 shows steps in an exemplary surgical method. In some alternative implementations, the functions noted in the various blocks may occur out of the order depicted in FIG. 6. For example, two blocks shown in succession in FIG. 6 may in fact be executed substantially concurrently, or the blocks may sometimes be executed in the reverse order. For this discussion, the embodiment with white and blue sutures will be described along with reference to FIG. 5.

First, as depicted in block 602, a cardiac patient is prepped for surgery. Then as depicted in block 604, the patient's chest cavity is exposed. A defective native heart valve is then removed as depicted in block 606 to expose a native valve annulus 32, and the artificial heart valve 30 is positioned near the native valve annulus as depicted in block 608 and as shown in FIG. 5.

To attach the artificial heart valve 30 to the native valve annulus 32, a suture 12 from the first set of sutures 10 is attached to the native valve annulus 32 as depicted in block 610 and FIG. 5 by passing both needles of the suture through the native valve annulus. The needles are then passed through a sewing ring 34 of the artificial valve 30. As depicted in block 612, a suture 22 from the second set of sutures 20 is then attached to the native valve annulus 32 in the same manner by passing both needles of the suture through the native valve annulus and then through the sewing ring 34 of the artificial valve 30. As depicted by blocks 614, 610, and 612, these steps are repeated with alternating sutures from the first and second sets of sutures such that adjacently positioned sutures are from different sets of sutures. After all of the sutures are placed, the loose ends of the sutures are tightened to firmly seat their pledgets against the native valve annulus and then tied as depicted in block 616. The surgery is then completed as depicted in block 618.

Advantageously, the alternating sutures allow the cardiac surgeon to more easily distinguish the sutures and determine which suture ends belong to which pledgets. Specifically, for the blue and white sutures in the embodiment described above, if the surgeon tightens the ends of a blue suture thread, the surgeon knows to look for and monitor the seating of a blue pledget and not the two adjacent white pledgets. Similarly, if the surgeon tightens the ends of a white suture thread, the surgeon knows to look for and monitor the seating of a white pledget and not the two adjacent blue pledgets. This not only prevents the above-described overtightening and undertightening problems but also allows surgeons to proceed more quickly by spending less time trying to match suture threads with their pledgets.

### Additional Considerations

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A surgical method for attaching an artificial heart valve to a native valve annulus, the surgical method comprising:
obtaining a first set of sutures, wherein each suture from the first set of sutures has a surgical thread with two ends, a first needle secured to the first end of the thread, a second needle attached to the second end of the thread, and a pledget, the thread and the pledget both having a first distinguishing feature;
obtaining a second set of sutures, wherein each suture from the second set of sutures has a surgical thread with two ends, a first needle secured to the first end of the thread, a second needle attached to the second end of the thread, and a pledget, the thread and the pledget both having a second distinguishing feature;
attaching a suture from the first set of sutures to the native valve annulus by passing the first and second needles of the suture through the native valve annulus then passing the first and second needles through a sewing ring of the artificial valve;
attaching a suture from the second set of sutures to the native valve annulus adjacent the suture from the first set of sutures by passing the first and second needles of the suture through the native valve annulus then passing the first and second needles through the sewing ring of the artificial valve;
repeating both the attaching steps with alternating sutures from the first and second sets of sutures such that adjacently positioned sutures are not from the same set of sutures;
after all of the sutures are placed, tightening the ends of the sutures to firmly seat the pledgets against the native valve annulus; and
tying the ends of the tightened sutures.

2. The surgical method of claim 1, wherein the first distinguishing feature is a first color.

3. The surgical method of claim 2, wherein the second distinguishing feature is a second color.

4. The surgical method of claim 3, wherein the first color is white.

5. The surgical method of claim 4, wherein the second color is blue.

6. The surgical method of claim 1, wherein the first distinguishing feature is a first pattern.

7. The surgical method of claim 2, wherein the second distinguishing feature is a second pattern.

8. The surgical method of claim 6, wherein the first pattern includes stripes.

9. The surgical method of claim 7, wherein the second pattern includes dots.

10. A surgical method for attaching a medical device to a human body part, the surgical method comprising:
obtaining a first set of sutures, wherein each suture from the first set of sutures has a surgical thread with two ends, a first needle secured to the first end of the thread, a second needle attached to the second end of the thread, and a pledget, the thread and the pledget both having a first distinguishing feature;
obtaining a second set of sutures, wherein each suture from the second set of sutures has a surgical thread with two ends, a first needle secured to the first end of the thread, a second needle attached to the second end of the thread, and a pledget, the thread and the pledget both having a second distinguishing feature;
attaching a suture from the first set of sutures to the human body part by passing the first and second needles of the suture through the human body part then passing the first and second needles through the medical device;
attaching a suture from the second set of sutures to the human body part adjacent the suture from the first set of sutures by passing the first and second needles of the suture through the human body part then passing the first and second needles through the medical device;
repeating both the attaching steps with alternating sutures from the first and second sets of sutures such that adjacently positioned sutures are not from the same set of sutures;
after all of the sutures are placed, tightening the ends of the sutures to firmly seat the pledgets against the human body part or the medical device; and
tying the ends of the tightened sutures.

11. The surgical method of claim 10, wherein the first distinguishing feature is a first color and the second distinguishing feature is a second color.

12. The surgical method of claim 11, wherein the first color is white and the second color is blue.

13. The surgical method of claim 10, wherein the first distinguishing feature is a first pattern and the second distinguishing feature is a second pattern.

14. The surgical method of claim 13, wherein the first pattern includes stripes and the second pattern includes dots.

15. A surgical method for attaching an artificial heart valve to a native valve annulus, the surgical method comprising:
obtaining a first set of sutures, wherein each suture from the first set of sutures has a surgical thread with two ends, a needle secured to one of the ends, and a pledget, the thread and the pledget both having a first distinguishing feature;
obtaining a second set of sutures, wherein each suture from the second set of sutures has a surgical thread with two ends, a needle secured one of the ends, and a pledget, the thread and the pledget both having a second distinguishing feature;
attaching a suture from the first set of sutures to the native valve annulus and the artificial valve;
attaching a suture from the second set of sutures to the native valve annulus and the artificial valve;
repeating both the attaching steps with alternating sutures from the first and second sets of sutures such that adjacently positioned sutures are not from the same set of sutures;
after all of the sutures are placed, tightening the ends of the sutures to firmly seat the pledgets against the native valve annulus; and
tying the ends of the tightened sutures.

16. The surgical method of claim 15, wherein the first distinguishing feature is a first color and the second distinguishing feature is a second color.

17. The surgical method of claim 16, wherein the first color is white and the second color is blue.

18. The surgical method of claim 15, wherein the first distinguishing feature is a first pattern and the second distinguishing feature is a second pattern.

19. A suture system for attaching an artificial heart valve to a native valve annulus, the suture system comprising:
a first set of sutures, each suture from the first set of sutures comprising:
a surgical thread with two ends,
a first needle secured to the first end of the thread,
a second needle attached to the second end of the thread, and
a pledget,
wherein the thread and the pledget both have a first distinguishing feature;
a second set of sutures, each suture from the second set of sutures comprising:
a surgical thread with two ends,
a first needle secured to the first end of the thread,
a second needle attached to the second end of the thread, and
a pledget,
wherein the thread and the pledget both have a second distinguishing feature;
a suture from the first set of sutures configured to be attached to the native valve annulus by passing the first and second needles of the suture through the native valve annulus then passing the first and second needles through a sewing ring of the artificial valve;
a suture from the second set of sutures configured to be attached to the native valve annulus adjacent the suture from the first set of sutures by passing the first and second needles of the suture through the native valve annulus then passing the first and second needles through the sewing ring of the artificial valve such that adjacently positioned sutures are not from the same set of sutures.

20. The suture system of claim 19, wherein the first distinguishing feature is a first color and the second distinguishing feature is a second color.
